(19) United States
(12) Patent Application Publication
(10) Pub. No.: US 2007/0049993 A1
(43) Pub. Date: Mar. 1, 2007

(54) DEVICE FOR PROTECTING AN ELECTRIC IMPEDANCE TOMOGRAPH FROM OVERVOLTAGE PULSES

(75) Inventors: Gerhard HOFMANN, Freiburg (DE); Jianhua LI, Lubeck (DE); Markus STEEGER, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 11/463,725

(22) Filed: Aug. 10, 2006

(30) Foreign Application Priority Data
Sep. 1, 2005 (DE) 10 2005 041 385.4

(51) Int. Cl. A61N 1/00

(52) U.S. Cl. 607/62

(57) ABSTRACT

A circuit is provided which protects the measuring input of an impedance tomograph from damage due to overvoltage. A resistor capacitor (RC) series connection is provided as a protective circuit at the measuring input.

### CROSS REFERENCE TO RELATED APPLICATIONS

This application claims the benefit of priority under 35 U.S.C. § 119 of German Patent Application DE 10 2005 041 385.4 filed Sep. 1, 2005, the entire contents of which are incorporated herein by reference.

### FIELD OF THE INVENTION

The present invention pertains to a device for protecting an electric impedance tomograph from overvoltage pulses.

### BACKGROUND OF THE INVENTION

Electrodiagnostic methods are frequently performed on patients who are in a critical state. It may become necessary in this connection to briefly use a defibrillator, without there being enough time to properly disconnect the patient from diagnostic devices. As a result, there is a risk of damage to the diagnostic devices due to overvoltage pulses.

Defibrillation is the only effective and life-saving procedure in life-threatening situations such as atrial fibrillation or pulseless ventricular tachycardia.

Any delay, which would arise due to the removal of electrodes or electric connections from the patient, is completely unacceptable.

According to the state of the art, input resistances of 10-50 kOhms are used in pure ECG (Electrocardiogram) devices or in combined ECG-impedance-measuring devices that are not used for imaging methods in order to prevent technical damage due to the use of defibrillators.

The special difficulty encountered in impedance tomographic methods is that in case of applications in thoracic electric impedance tomography, the electrodes are frequently intended for a dual purpose, contrary to pure electrocardiography.

Firstly, they shall introduce into the patient the excitation currents, which may reach up to 10 mA and with which a readily evaluable potential distribution is to be obtained in the patient.

Secondly, they shall again send the weak signal currents, which are measured on the skin surface of the patient on the basis of the potential distribution generated with the excitation currents, to the input amplifier. The signal currents to be measured may be in the nanoampere range.

Thus, the currents to be introduced must be selected to be high, with values of up to 10 mA, in order to generate sufficient potential differences in the entire thorax to make it possible to generate an image of the potentials picked up. Voltages of 100-500 V would be obtained over resistances of 10-50 kOhms. Such voltages cannot be used on the patient, and the possibility of securing the impedance tomograph by sufficiently high drop resistors cannot therefore be considered.

Securing the inputs by varistors or diodes connected in parallel to the input amplifier is likewise problematic. Additional stray capacitances, which are connected between the signal line and the reference potential, must be kept as low as possible. This is necessary to prevent unacceptable reactive impedances, which are in parallel to the input of the first amplifier stage, from forming at the usual working frequencies of about 10-200 kHz. They would unacceptably increase the load, which the measuring circuit represents in relation to the potential distribution on the skin surface of the patient and thus distort the measurement. At a frequency of 50 kHz, even 10 pF represent an impedance of about 30 kOhms. Solutions that contain varistors or diodes connected in parallel to the input amplifier are thus ruled out if their interference capacitance is greater than a few pF. However, all types that could dissipate the currents that are usually generated by a defibrillator shock are eliminated as a result.

Therefore, it must be assumed that the defibrillator is used without the patient being disconnected from the impedance tomograph. Besides the necessary protection of the circuit from overload, it is necessary to avoid excessive draining of the energy of the defibrillator shock in order not to unacceptably limit the effectiveness of the defibrillator.

For example, standards require that a maximum of 10% of the energy of a defibrillator pulse may be dissipated by the measuring circuit if sufficient effectiveness shall still be assumed. Equivalent standard requirements for thoracic impedance tomographs are undoubtedly to be expected in case this diagnostic method becomes established. Effective use of the defibrillator must be guaranteed for the protection of the patient before the protection of device components that may be at risk.

### SUMMARY OF THE INVENTION

The object of the present invention is to provide a device that reliably protects a thoracic electric impedance tomograph connected to a patient from damage due to overvoltage in case of the use of a defibrillator or electrocauter, without there being a risk that the energy of the defibrillator pulses is drained off to such an extent that it is detrimental to the effectiveness of defibrillation, and the ability of the impedance tomograph to function shall be preserved.

The object is accomplished by providing an electric impedance tomograph where the tomograph's signal inputs are provided with a protective circuit that secures the signal inputs from excessively high input currents when a voltage is too high for the normal measuring operation is present.

The basic idea of the present invention is based on the distinction between two operating states:

1. The normal operation, in which both currents for excitation in the range of 1-10 mA and currents for detection in the range of typically 100 nA to 5 μA flow through the electrode connections.

2. The extreme state, in which voltages that exceed the supply voltage and may reach up to 5 kV are admitted to the electrodes ab externo.

Thus, the present invention comprises an electric impedance tomograph, whose signal inputs are provided with a protective circuit, which secures the signal inputs from high input currents when a voltage that is too high for the normal measuring operation is present. This happens such that the effectiveness of defibrillation is ensured and the device is protected from overvoltage pulses.

A low-ohmic resistor, which is typically in the range below 1 kOhm, is used during the normal operation of the impedance tomograph. In case of loading by an excessively high voltage during defibrillation, the energy uptake is limited by effective measures via the circuit of the impedance tomograph in a sufficiently short time to a sufficiently great extent.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 shows a schematic view of the present invention for protecting an electric impedance tomograph from overvoltage pulses.

FIG. 2 shows a schematic view of the present invention with a plurality of electrodes placed within an electrode belt

### DESCRIPTION OF THE PREFERRED EMBODIMENT

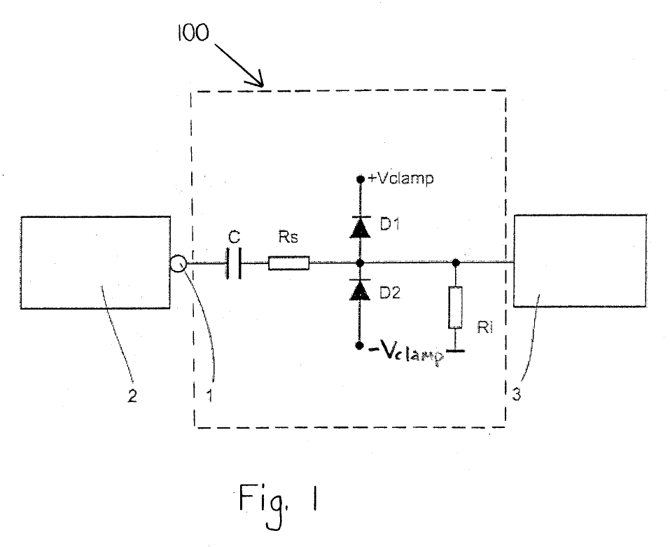

Referring to the FIG. 1, the electric impedance tomography (EIT) device includes a protective circuit 100, which comprises a resistor capacitor (RC) series connection between an electrode 1, which can be connected to a patient 2, and the input (signal input) of the EIT device 3. The combination of a series-connected high-voltage capacitor C and a resistor Rs at the input of the impedance tomograph, has a low impedance during normal operation because of the frequencies used for the current feed, typically 25-200 kHz here. The diodes D1 and D2 are blocked during normal operation.

The potentials resulting from the current feed at the other electrodes can be measured without marked attenuation because the impedance of the RC series connection compared to the input impedance of the impedance tomograph is very low. The diodes D1 and D2 are also blocked during the measurement of the potentials in normal operation.

High voltage, up to 5 kV in the extreme case, is present on the electrode during defibrillation. The energy absorbed by the protective circuit 100 is limited by the capacitor C, which permits a very brief loading current only. The resistor Rs limits the loading current, which could otherwise become too high in case of a very rapid rise of a defibrillator pulse.

During the positive flank of the defibrillator pulse, the loading current is drained off via D1 to the supply voltage +V_{camp} as soon as the potential at the node between D1 and D2 exceeds +V_{camp}. The energy drained off is absorbed in the voltage supply. The impedance tomograph is protected in this manner. As soon as the potential drops on the electrode, the energy being stored is discharged via D2. The discharge current flows over the diode D2 to the supply voltage −V_{camp} as soon as the potential at the node between D1 and D2 is below −V_{camp}. The potential at the input of the impedance tomograph will thus always be between +V_{camp} and −V_{camp}, as a result of which the input is reliably protected from high-voltage pulses. The majority of the energy of the defibrillator pulses is in the low-frequency range. The energy loss is limited by selecting a suitable capacitance.

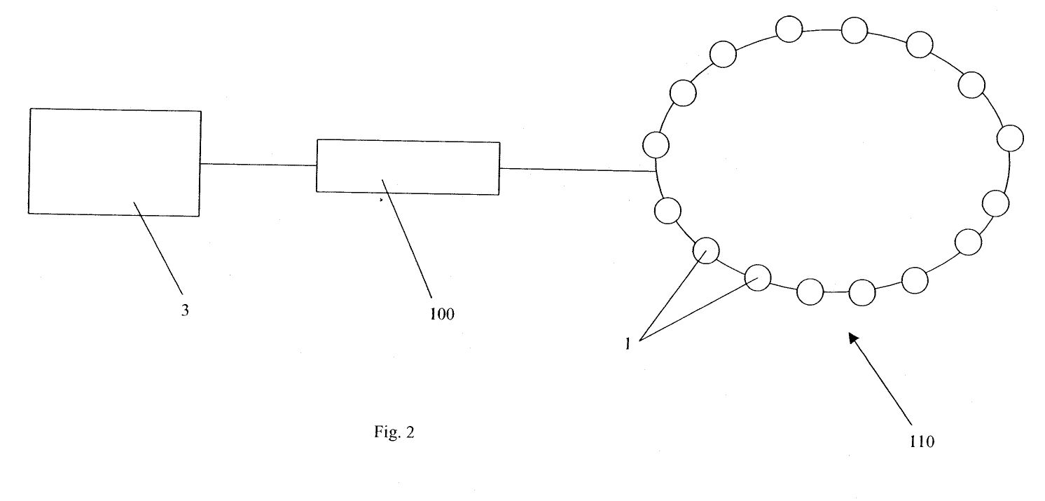

Referring to FIG. 2, more than one electrode 1 may be advantageously provided in an electrode belt 110. The electrode belt 110 may be connected to the protective circuit 100 which is connected to the electric impedance tomography 3. Each electrode 1 may advantageously individually wired to the protective circuit 100.

The percentage of absorbed energy is nearly independent from the selected energy level of the defibrillator.

The shielding of measuring lines used can be additionally secured with such a protective circuit.

Various possibilities are available for integrating the protective circuit according to the present invention in EIT devices. Thus, the protective circuit may advantageously be integrated in the electrodes used with the impedance tomograph.

Furthermore, it may be advantageous to integrate the protective circuit in an electrode carrier used with the impedance tomograph.

Furthermore, the protective circuit may be integrated in electric plug-type connections used with the impedance tomograph or designed as a part of electrode cables used with the impedance tomograph.

While a specific embodiment of the invention has been shown and described in detail to illustrate the application of the principles of the invention, it will be understood that the invention may be embodied otherwise without departing from such principles.

What is claimed:

1. An electric impedance tomograph comprising:
electric impedance tomograph signal input;
an electrode for connection to a patient; and
a voltage protection circuit connected to said electrode and to said signal input, said voltage protection circuit shielding said signal inputs from high input currents that are too high for normal measuring operation.

2. An electric impedance tomograph in accordance with claim 1, further comprising:
measuring channels, each said measuring channel being equipped with a protective circuit to minimize overvoltage pulses.

3. An electric impedance tomograph in accordance with claim 2, wherein said protective circuit comprises a resistor capacitor (RC) series connection contained within said measuring channels.

4. An electric impedance tomograph in accordance with claim 1, further comprising:
shieldings of measuring lines, said shieldings being equipped with a protective circuit.

5. An electric impedance tomograph in accordance with claim 1, wherein said protective circuit is integrated in electrodes.

6. An electric impedance tomograph in accordance with claim 1, wherein said protective circuit is integrated in an electrode carrier.

7. An electric impedance tomograph in accordance with claim 1, wherein said protective circuit is integrated in electric plug-type connections.

8. An electric impedance tomograph in accordance with claim 1, wherein said protective circuit is integrated in electrode cables.

9. An electric impedance tomograph comprising:
at least one electric impedance tomograph signal input means;
a plurality of electrodes for connection to a patient;
a voltage protection circuit connected to at least one electrode and to said signal input, said voltage protection circuit shielding said signal inputs from high input currents that are too high for normal measuring operation, said signal input means for passing a signal from at least one electric impedance tomograph signal input through at least one voltage protection circuit to at least one electrode and detecting a feedback signal passing from at least one electrode sent through said voltage protection circuit to at least one electrical impedance signal input and providing the feedback signal for further processing to create an image.

10. An electric impedance tomograph in accordance with claim 9, further comprising:
measuring channels, wherein each said measuring channel is equipped with a voltage protection circuit to minimize overvoltage pulses.

11. An electric impedance tomograph in accordance with claim 10, wherein said voltage protection circuit comprises a resistor capacitor (RC) series connection contained within said measuring channels.

12. An electric impedance tomograph in accordance with claim 9, further comprising:
shieldings of measuring lines, said shieldings being equipped with a voltage protection circuit.

13. An electric impedance tomograph in accordance with claim 9, wherein said voltage protection circuit is integrated within electrodes used with an impedance tomograph.

14. An electric impedance tomograph in accordance with claim 9, wherein said voltage protection circuit is integrated in an electrode carrier used with an impedance tomograph.

15. An electric impedance tomograph in accordance with claim 9, wherein said voltage protection circuit is integrated in electric plug-type connections used with an impedance tomograph.

16. An electric impedance tomograph in accordance with claim 9, wherein said voltage protection circuit is integrated in electrode cables used with the impedance tomograph.

17. An electric impedance tomograph comprising:
electric impedance tomograph signal input;
an electrode connected to a patient;
an overvoltage shielding circuit connected to said electrode and to said electric impedance tomograph signal input, wherein said overvoltage shielding circuit detects high input currents that are too high for normal measuring operation and dissipates the high input current to protect said electric impedance signal input from the high input currents.

18. An electric impedance tomograph in accordance with claim 17, wherein said overvoltage shielding circuit includes a voltage supply.

19. An electric impedance tomograph in accordance with claim 18, wherein said overvoltage shielding circuit dissipates the high input currents to said voltage supply.

20. A device in accordance with claim 17, further comprising:
measuring channels, wherein each said measuring channel is equipped with an overvoltage shielding circuit to minimize overvoltage pulses.
